(19) United States
(12) Patent Application Publication
(10) Pub. No.: US 2024/0325626 A1
(43) Pub. Date: Oct. 3, 2024

(54) Attachable IV Manifold Device

(71) Applicant: Joseph Bettencourt, Loudon, TN (US)

(72) Inventor: Joseph Bettencourt, Loudon, TN (US)

(21) Appl. No.: 18/471,530

(22) Filed: Sep. 21, 2023

Related U.S. Application Data
(60) Provisional application No. 63/492,502, filed on Mar. 28, 2023.

(51) Int. Cl. A61M 5/14

(52) U.S. Cl. CPC A61M 5/1418; A61M 5/1415

(57) ABSTRACT

An attachable IV manifold device is provided. The device is primarily comprised of at least one IV manifold comprised of a main body with at least one opening, at least one port, and at least one fastener. The device is designed to allow an IV manifold to be easily attached to nearby surfaces, structures, objects, etc., via the fastener which prevents the manifold from falling onto the floor, shifting in position, becoming tangled, etc., during use.

### CROSS-REFERENCE TO RELATED APPLICATION

The present application claims priority to, and the benefit of, U.S. Provisional Application No. 63/492,502, which was filed on Mar. 28, 2023, and is incorporated herein by reference in its entirety.

### FIELD OF THE INVENTION

The present invention relates generally to the field of IV manifolds. More specifically, the present invention relates to an attachable IV manifold device comprised of an IV manifold and a fastener which allows the manifold to be attached to a surface, object, structure, etc. Accordingly, the present disclosure makes specific reference thereto. Nonetheless, it is to be appreciated that aspects of the present invention are also equally applicable to other like applications, devices, and methods of manufacture.

### BACKGROUND

IV injection port manifolds play a crucial role in the administration of medications and compounds to patients through intravenous (IV) routes. These manifolds serve as a conduit to deliver necessary substances and regulate pressure accurately. However, IV manifolds may be inadvertently bumped, leading to their displacement onto the floor. This poses a serious health risk as it exposes the manifold to potential contamination. Any contaminants that make their way into the manifold could ultimately find their way into the patient's bloodstream, potentially causing harm or infection.

Another issue associated with the placement of IV tubing from the manifold is the creation of a tripping hazard. Healthcare environments can be bustling with activity, and the presence of tubing across the floor increases the likelihood of accidents and falls. The consequences of tripping over the tubing can range from minor injuries to more severe complications, impacting patients and healthcare providers. Furthermore, if a manifold falls due to mishandling or accidental contact, there is a possibility that it could dislodge an IV catheter from the patient's vein. This unintended removal of the catheter can result in blood loss from the open vein, necessitating prompt intervention to address the situation.

To address these challenges, healthcare professionals have often resorted to a common workaround: taping the injection port manifold to a patient's bedsheets. However, this solution is far from perfect and introduces its own set of concerns. The attachment achieved through tape is inherently insecure and prone to becoming dislodged or loosened over time. Consequently, the manifold may become unstable, jeopardizing the integrity of the IV line and potentially causing interruptions in medication delivery or other complications.

Moreover, even with the manifold taped securely, there remains a risk of inadvertently pulling out the IV catheter if the healthcare provider forgets to remove the tape from the manifold before disconnecting it. This could result in not only the dislodgment of the catheter but also potential discomfort or pain for the patient during the removal process. These challenges highlight the need for a more reliable and user-friendly solution that can address the secure attachment of IV injection port manifolds while mitigating the associated risks of contamination, tripping hazards, and accidental catheter removal.

Therefore, there exists a long-felt need in the art for an IV manifold device. More specifically, there exists a long-felt need in the art for an attachable IV manifold device that allows a user to attach an IV manifold. Further, there exists a long-felt need in the art for an attachable IV manifold device that allows a user to attach an IV manifold to a structure, surface, or object such that the manifold cannot be easily dislodged. In addition, there exists a long-felt need in the art for an attachable IV manifold device that allows a user to attach an IV manifold to a structure, surface, or object such that the manifold cannot be easily dislodged and wherein the manifold can be positioned as desired to route IV lines.

The subject matter disclosed and claimed herein, in one embodiment thereof, comprises an attachable IV manifold device. The device is primarily comprised of at least one IV manifold comprised of a main body with at least one opening, at least one port, and at least one fastener. The device is designed to allow an IV manifold to be easily attached to nearby surfaces, structures, objects, etc., via the fastener. This prevents the manifold from falling onto the floor, shifting in position, becoming tangled, etc., during use. In the preferred embodiment, the fastener is comprised of a spring-loaded clip or clamp.

In this manner, the attachable IV manifold device of the present invention accomplishes all the foregoing objectives and provides a device that allows a user to attach an IV manifold to a structure, surface, or object such that the manifold cannot be easily dislodged. In addition, the device allows a user to attach an IV manifold to a structure, surface, or object such that the manifold cannot be easily dislodged and wherein the manifold can be positioned as desired to route IV lines.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an attachable IV manifold device. The device is primarily comprised of at least one IV manifold comprised of a main body with at least one opening, at least one port, and at least one fastener. The device is designed to allow an IV manifold to be easily attached to nearby surfaces, structures, objects, etc., via the fastener. This prevents the manifold from falling onto the floor, shifting in position, becoming tangled, etc., during use.

More specifically, the manifold is comprised of a main body with a first opening and a second opening that are in fluid communication. Each opening can receive at least one IV line or other medical tubing that travels to/from a patient. The body is also comprised of at least one port that allows at least one additional IV line to connect to the main body to push various medications, compounds, pressure, etc., into the main body via at least one opening of the port. Then, the medication, compound, etc., can be pushed to the patient via the IV line leaving the first or second opening of the main body. The port may be comprised of at least one stopcock valve that inhibits or allows flow from the port to the main body in at least one direction. Said valve may also be positioned near the first opening and/or second opening to allow or inhibit flow from each opening to/from the patient.

The main body is also comprised of at least one fastener that may fixedly or removably attach to the main body in different embodiments. In a removable embodiment, at least one receiving member attaches to the main body. In a fixedly attached embodiment, the fastener is integrally formed to the main body. The fastener is preferably a clip or clamp comprised of at least one spring-loaded hinge that allows a set of jaws of the fastener to open/close via pulling or releasing at least one handle. This allows the jaws to be attached around any surface, structure, object, etc., such as, but not limited to, an IV pole, a hospital bed, etc., which secures the device and prevents it from falling onto the ground, shifting position, etc.

The present invention is also comprised of a first method of using the device. First, a device is provided comprised of a main body comprised of at least one opening, at least one port with at least one opening attached to and in fluid communication with the main body, and at least one fastener integrally formed to the main body. Then, at least one jaw of the fastener can be attached to/around a surface. Next, a first IV line can be attached to a first opening of the main body and a second IV line can be attached to a second opening of the main body. Then, a third IV line can be attached to the port. Finally, at least one stopcock of the port and/or openings can be opened or closed to allow or prohibit medication, pressure, or other compounds to enter the main body from the third IV line.

The present invention is also comprised of a second method of using the device. First, a device is provided comprised of a main body comprised of at least one opening, at least one port with at least one opening attached to and in fluid communication with the main body, and at least one fastener that removably attaches to the main body. Next, at least one receiving member of the fastener can be attached to the main body to secure the fastener to the main body. Then, at least one jaw of the fastener can be attached to/around a surface. Next, a first IV line can be attached to a first opening of the main body and a second IV line can be attached to a second opening of the main body. Then, a third IV line can be attached to the port. Finally, at least one stopcock of the port and/or openings can be opened or closed to allow or prohibit medication, pressure, or other compounds to enter the main body from the third IV line.

Accordingly, the attachable IV manifold device of the present invention is particularly advantageous as it provides a device that allows a user to attach an IV manifold. More specifically, the device allows a user to attach an IV manifold to a structure, surface, or object such that the manifold cannot be easily dislodged. In addition, the device allows a user to attach an IV manifold to a structure, surface, or object such that the manifold cannot be easily dislodged and wherein the manifold can be positioned as desired to route IV lines. In this manner, the attachable IV manifold device overcomes the limitations of existing methods of attaching IV manifolds known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an attachable IV manifold device of the present invention in accordance with the disclosed architecture;

FIG. 2 illustrates an enhanced perspective view of a fastener of one potential embodiment of an attachable IV manifold device of the present invention in accordance with the disclosed architecture;

FIG. 3 illustrates a perspective view of one potential embodiment of an attachable IV manifold device of the present invention while attached to a hospital bed in accordance with the disclosed architecture;

FIG. 4 illustrates a flowchart of a method of using one potential embodiment of an attachable IV manifold device of the present invention in accordance with the disclosed architecture; and

FIG. 5 illustrates a flowchart of a method of using one potential embodiment of an attachable IV manifold device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an IV manifold device. More specifically, there exists a long-felt need in the art for an attachable IV manifold device that allows a user to attach an IV manifold. Further, there exists a long-felt need in the art for an attachable IV manifold device that allows a user to attach an IV manifold to a structure, surface, or object such that the manifold cannot be easily dislodged. In addition, there exists a long-felt need in the art for an attachable IV manifold device that allows a user to attach an IV manifold to a structure, surface, or object such that the manifold cannot be easily dislodged and wherein the manifold can be positioned as desired to route IV lines.

The present invention, in one exemplary embodiment, is comprised of an attachable IV manifold device primarily comprised of at least one IV manifold comprised of a main body with at least one opening, at least one port, and at least one fastener. The device is designed to allow an IV manifold to be easily attached to nearby surfaces, structures, objects, etc., via the fastener to prevent the manifold from falling onto the floor, shifting in position, becoming tangled, etc., during use.

More specifically, the manifold is comprised of a main body with a first opening and a second opening that are in fluid communication. Each opening can receive at least one IV line or other medical tubing that travels to/from a patient. The body is also comprised of at least one port that allows at least one additional IV line to connect to the main body to push various medications, compounds, pressure, etc., into the main body via at least one opening of the port. Then, the medication, compound, etc., can be pushed to the patient via the IV line leaving the first or second opening of the main body. The port may be comprised of at least one stopcock valve. The valve inhibits or allows flow from the port to the main body in at least one direction. Said valve may also be positioned near the first opening and/or second opening to allow or inhibit flow from each opening to/from the patient.

The main body is also comprised of at least one fastener. The fastener may fixedly or removably attach to the main body in different embodiments. In a removable embodiment, at least one receiving member attaches to the main body. In a fixedly attached embodiment, the fastener is integrally formed to the main body. The fastener is preferably a clip or clamp comprised of at least one spring-loaded hinge that allows a set of jaws of the fastener to open/close via pulling or releasing at least one handle. This allows the jaws to be attached around any surface, structure, object, etc., such as, but not limited to, an IV pole, a hospital bed, etc., which secures the device and prevents it from falling onto the ground, shifting position, etc.

The present invention is also comprised of a first method of using the device. First, a device is provided comprised of a main body comprised of at least one opening, at least one port with at least one opening attached to and in fluid communication with the main body, and at least one fastener integrally formed to the main body. Then, at least one jaw of the fastener can be attached to/around a surface. Next, a first IV line can be attached to a first opening of the main body and a second IV line can be attached to a second opening of the main body. Then, a third IV line can be attached to the port. Finally, at least one stopcock of the port and/or openings can be opened or closed to allow or prohibit medication, pressure, or other compounds to enter the main body from the third IV line.

The present invention is also comprised of a second method of using the device. First, a device is provided comprised of a main body comprised of at least one opening, at least one port with at least one opening attached to and in fluid communication with the main body, and at least one fastener that removably attaches to the main body. Next, at least one receiving member of the fastener can be attached to the main body to secure the fastener to the main body. Then, at least one jaw of the fastener can be attached to/around a surface. Next, a first IV line can be attached to a first opening of the main body and a second IV line can be attached to a second opening of the main body. Then, a third IV line can be attached to the port. Finally, at least one stopcock of the port and/or openings can be opened or closed to allow or prohibit medication, pressure, or other compounds to enter the main body from the third IV line.

Accordingly, the attachable IV manifold device of the present invention is particularly advantageous as it provides a device that allows a user to attach an IV manifold. More specifically, the device allows a user to attach an IV manifold to a structure, surface, or object such that the manifold cannot be easily dislodged. In addition, the device allows a user to attach an IV manifold to a structure, surface, or object such that the manifold cannot be easily dislodged and wherein the manifold can be positioned as desired to route IV lines. In this manner, the attachable IV manifold device overcomes the limitations of existing methods of attaching IV manifolds known in the art.

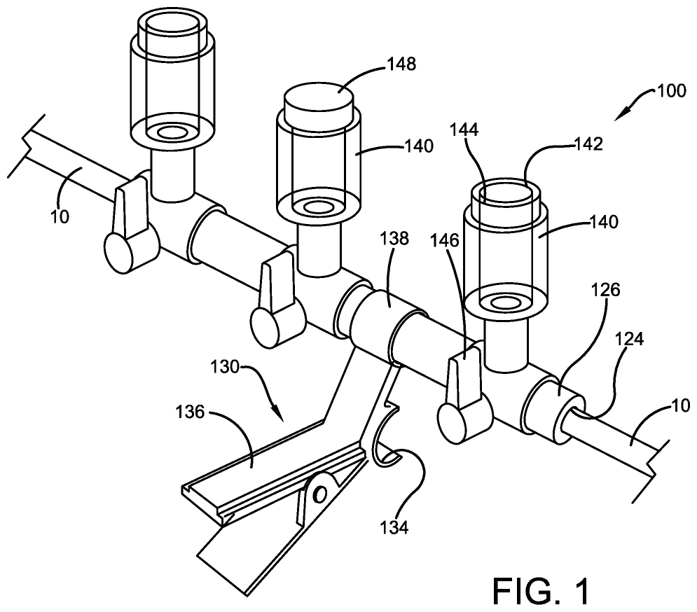

Referring initially to the drawings, FIG. 1 illustrates a perspective view of one potential embodiment of an attachable IV manifold device 100 of the present invention in accordance with the disclosed architecture. The device 100 is primarily comprised of at least one IV manifold 110 comprised of a main body 120 with at least one opening 122,124, at least one port 140, and at least one fastener 130. The device 100 is designed to allow an IV manifold 110 to be easily attached to nearby surfaces 12, structures, objects, etc., via the fastener 130. This prevents the manifold 110 from falling onto the floor, shifting in position, becoming tangled, etc., during use. The manifold 110 may be the shape, size, and configuration of any style of IV manifold (or similar structure) that allows the manifold 110 to push multiple medications, compounds, pressure, etc., from multiple IV lines 10 using one manifold 110.

More specifically, the manifold 110 is comprised of a main body 120. The main body 120 is preferably tubular and hollow to allow for fluid to travel through the body 120. The body 120 has a first opening 122 and a second opening 124 that are in fluid communication. Each opening 122,124 can each receive at least one IV line 10 or other medical tubing that travels to/from a patient. The openings 122,124 may be comprised of any attachment structure 126 known in the art that allows the line 10 to removably attach to each opening 122,124.

The body 110 is also comprised of at least one port 140. The port 140 allows at least one additional IV line 10 to connect to the main body 120 (via at least one attachment member 144 of any style of IV line 10 attachment known in the art) to push various medications, compounds, pressure, etc., into the main body 120 via at least one opening 142 of the port 140. Then, the medication, compound, etc., can be pushed to the patient via the IV line 10 leaving the first or second opening 122,124. The port 140 may be any style of IV port.

Preferably, the port 140 is comprised of at least one stopcock valve 146 (or other structure of the like) that inhibits or allows flow from the port 140 to the main body 120 in at least one direction. When not in use, the port 140 can receive at least one cap 148 to cover the opening 142. Said valve 146 may also be positioned near the first opening 122 and/or second opening 124 to allow or inhibit flow from each opening 122,124 to/from the patient. Said openings 122,124 may also be covered with a cap 148 when not in use.

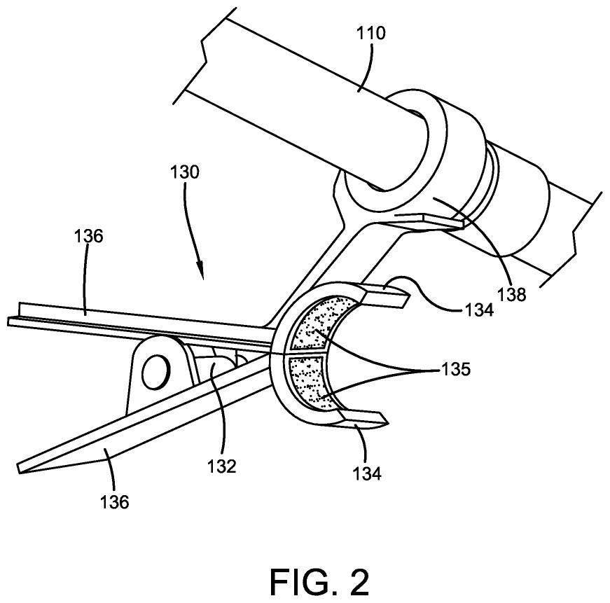

The main body 120 is also comprised of at least one fastener 130, as seen in FIG. 2. The fastener 130 may fixedly or removably attach to the main body 120 in different embodiments. In a removable embodiment, at least one receiving member 138 attaches to the main body 120. The member 138 may be any style of removable attachment member known in the art such as, but not limited to, a clip, a clamp, a snap fastener, etc. In a fixedly attached embodiment, the fastener 130 is integrally formed to the main body 120.

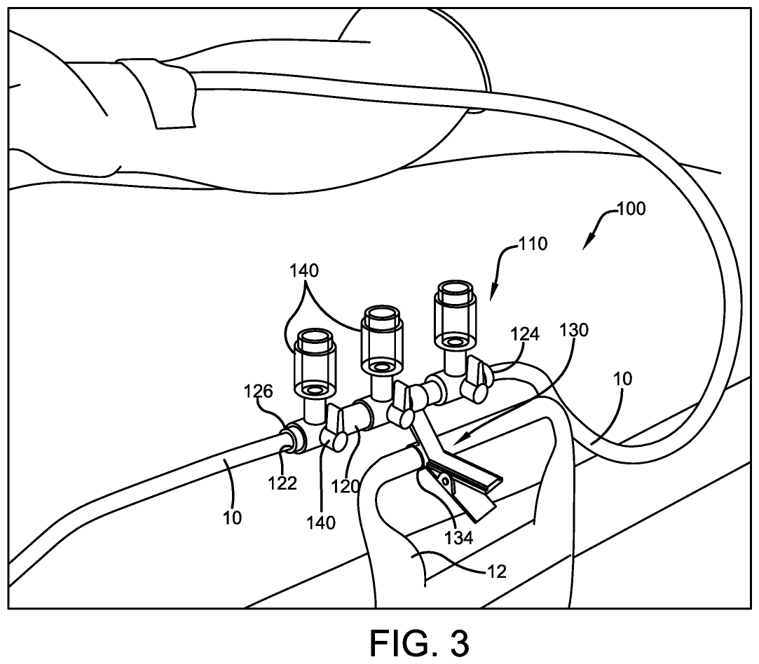

The fastener 130 is preferably a clip or clamp. In said preferred embodiment, the fastener 130 is comprised of at least one spring-loaded hinge 132 that allows a set of jaws 134 of the fastener 130 to open/close via pulling or releasing at least one handle 136. This allows the jaws 134 to be attached around any surface 10, structure, object, etc., such as, but not limited to, an IV pole, a hospital bed, etc., which secures the device 100 and prevents it from falling onto the ground, shifting position, etc., as seen in FIG. 3.

In one embodiment, the jaws 134 may be comprised of at least one non-slip grip area 135 to improve the grip of the jaws 134 on a surface 10. The grip area 135 may be made from a non-slip material such as, but not limited to rubber, silicone, etc. The grip area 135 may also have a raised or recessed grip texture to improve grip.

However, it should be appreciated that the fastener 130 may be different fastener types in different embodiments, such as, but not limited to, magnet, snap fastener, hook and loop, clip, clamp, locking fastener, etc. In addition, all components of the device 100 may be transparent to allow a user to view medications, compounds, etc., moving through the device 100 during use.

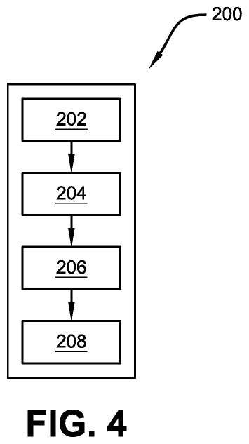

The present invention is also comprised of a first method of using 200 the device 100, as seen in FIG. 4. First, a device 100 is provided comprised of a main body 120 comprised of at least one opening 122,124, at least one port 140 with at least one opening 142 attached to and in fluid communication with the main body 120, and at least one fastener 130 integrally formed to the main body 120 [Step 202]. Then, at least one jaw 134 of the fastener 130 can be attached to/around a surface 10 [Step 204]. Next, a first IV line 10 can be attached to a first opening 122 of the main body 120 and a second IV line 10 can be attached to a second opening 124 of the main body 120 [Step 206]. Then, a third IV line 10 can be attached to the port 140 [Step 208]. Finally, at least one stopcock 146 of the port 140 and/or openings 122,124 can be opened or closed to allow or prohibit medication, pressure, or other compounds to enter the main body 120 from the third IV line 10 [Step 210].

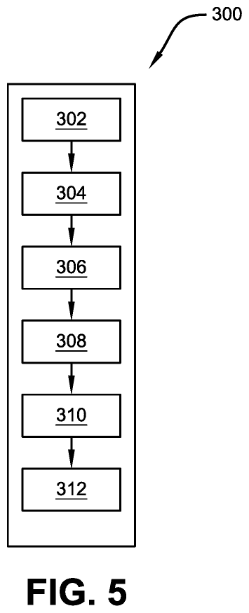

The present invention is also comprised of a second method of using 300 the device 100, as seen in FIG. 5. First, a device 100 is provided comprised of a main body 120 comprised of at least one opening 122,124, at least one port 140 with at least one opening 142 attached to and in fluid communication with the main body 120, and at least one fastener 130 that removably attaches to the main body 120 [Step 302]. Next, at least one receiving member 138 of the fastener 130 can be attached to the main body 120 to secure the fastener 130 to the main body [Step 304]. Then, at least one jaw 134 of the fastener 130 can be attached to/around a surface 10 [Step 306]. Next, a first IV line 10 can be attached to a first opening 122 of the main body 120 and a second IV line 10 can be attached to a second opening 124 of the main body 120 [Step 308]. Then, a third IV line 10 can be attached to the port 140 [Step 310]. Finally, at least one stopcock 146 of the port 140 and/or openings 122,124 can be opened or closed to allow or prohibit medication, pressure, or other compounds to enter the main body 120 from the third IV line 10 [Step 312].

Certain terms are used throughout the following description and claims to refer to particular features or components. As one skilled in the art will appreciate, different persons, may refer to the same feature or component by different names. This document does not intend to distinguish between components or features that differ in name but not structure or function. As used herein “attachable IV manifold device” and “device” are interchangeable and refer to the attachable IV manifold device 100 of the present invention.

Notwithstanding the foregoing, the attachable IV manifold device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the attachable IV manifold device 100 as shown in the FIGS. are for illustrative purposes only, and many other sizes and shapes of the attachable IV manifold device 100 are well within the scope of the present disclosure. Although the dimensions of the attachable IV manifold device 100 are important design parameters for user convenience, the attachable IV manifold device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An attachable IV manifold device comprising:
a main body comprised of a first opening;
a port comprised of a second opening, the port positioned on and in fluid communication with the main body;
a first valve; and
a fastener comprised of a receiving member.

2. An attachable IV manifold device of claim 1, wherein the receiving member attaches to the main body.

3. An attachable IV manifold device of claim 1, wherein the receiving member is comprised of a snap fastener.

4. An attachable IV manifold device of claim 1, wherein the first valve is comprised of a stopcock valve.

5. An attachable IV manifold device of claim 1, wherein the first opening is comprised of a second valve.

6. An attachable IV manifold device of claim 5, wherein the second valve is comprised of a stopcock valve.

7. An attachable IV manifold device comprising:
a main body comprised of a first opening;
a port comprised of a second opening, the port positioned on and in fluid communication with the main body;
a valve; and
a fastener fixedly attached to the main body.

8. An attachable IV manifold device of claim 7, wherein the fastener is comprised of a clip or a clamp.

9. An attachable IV manifold device of claim 8, wherein the fastener is comprised of a spring-loaded hinge.

10. An attachable IV manifold device of claim 7, wherein the fastener is comprised of a pair of jaws.

11. An attachable IV manifold device of claim 10, wherein the pair of jaws are each comprised of a grip area.

12. An attachable IV manifold device of claim 11, wherein the grip area is comprised of a non-slip material.

13. An attachable IV manifold device of claim 11, wherein the grip area is comprised of a raised or a recessed texture.

14. An attachable IV manifold device of claim 7, wherein the fastener is comprised of a handle.

15. An attachable IV manifold device of claim 7, wherein the port is comprised of a cap.

16. An attachable IV manifold device of claim 7, wherein the port is comprised of a first attachment member.

17. An attachable IV manifold device of claim 7, wherein the first opening is comprised of a second attachment member.

18. A method of using an attachable IV manifold device, the method comprising the following steps:
providing an attachable IV manifold device comprised of a fastener, and a main body comprised of a first opening and a port;
attaching the fastener to a surface;
attaching a first IV line to the first opening;
attaching a second IV line a second opening of the main body; and
attaching a third IV line to the port.

19. A method of using an attachable IV manifold device of claim 18 further comprised of a step of opening or closing a valve of the main body or the port.

20. A method of using an attachable IV manifold device of claim 18 further comprised of a step of attaching the fastener to the main body.
